(19) United States
(12) Patent Application Publication
(10) Pub. No.: US 2008/0115243 A1
(43) Pub. Date: May 15, 2008

(54) Transgenic Plants Expressing Intein Modified Proteins and Associated Processes for Bio-Pharmaceutical Production

(75) Inventors: R. Michael Raab, Cambridge, MA (US); Kyle L. Jensen, Cambridge, MA (US); Karl Ruping, Cambridge, MA (US)

Correspondence Address: VOLPE AND KOENIG, P.C., UNITED PLAZA, SUITE 1600, 30 SOUTH 17TH STREET, PHILADELPHIA, PA, 19103, US

(73) Assignee: Agrivida, Inc., Cambridge, MA (US)

(21) Appl. No.: 11/596,821

(22) PCT Filed: May 19, 2005
(86) PCT No.: PCT/US05/17700

§ 371 Date: Aug. 20, 2007

Related U.S. Application Data
(60) Provisional application No. 60/572,966, filed on May 19, 2004.

(51) Int. Cl. C12N 15/87; A01H 1/00; C12P 21/04; C12N 5/00

(52) U.S. Cl. 800/288; 800/298; 435/410; 435/470; 435/69.1

(57) ABSTRACT

Transgenic plants that express CIVPS or intein modified therapeutic proteins, compositions of matter comprising them, therapeutic proteins made from the transgenic plants, methods to construct the transgenic plants containing CIVPS or intein modified therapeutic genes, methods to express CIVPS or intein modified therapeutic proteins in plants, and methods of using the transgenic plants.

### FIELD OF INVENTION

The present invention relates to transgenic plants expressing CIVPS or intein fused polypeptides from therapeutic proteins, methods for the production of the transgenic plants, methods for the expression of controllable intervening protein sequences (“CIVPS”) or intein modified proteins in plants, processes for producing therapeutic proteins from the plants, and various uses of and products containing the transgenic plants expressing CIVPS or intein modified proteins.

### BACKGROUND OF THE INVENTION

The pharmaceutical industry is dependent upon a consistent supply of proteins that have specific therapeutic properties, called therapeutic proteins, for a new generation of drugs derived from advances in biotechnology research. Unlike traditional medicines that may be synthetically produced, therapeutic proteins are usually produced through microbial fermentation or by mammalian cell culture.

In mammalian cell culture and microbial fermentation, cells are grown in large fermentation tanks, vats, or containers. The cells are kept alive and stimulated to produce the target proteins under precise environmental conditions such as temperature, oxygen level, and acidity. The proteins are then isolated and purified from the cultures, and finally formulated into the final pharmaceutical products.

Therapeutic protein production in plants has a number of advantages that make it an attractive alternative to traditional cell culture and fermentation processes. The operating costs for protein production using plants are estimated to be ten-fold less per gram than for cell culture or fermentation processes. In addition, the capital costs of manufacturing facilities to produce proteins from plants are significantly less than the capital required for traditional culturing processes due to the elimination of the large-scale, up-stream culturing suite. Plant protein production also has advantages over microbial production because plants can properly fold complex proteins, do not normally produce inclusion bodies, and glycosylation is possible in plants while totally absent in bacteria. Unlike mammalian cell culture systems that can also fold and glycosylate proteins properly, plants typically do not harbor human infectious agents that can potentially contaminate cell culture systems providing another layer of safety. Scaling up protein production in plants is also simple compared to the research and effort involved in scaling up cell culture systems which require special containment and sterility provisions over long periods of time.

There are a number of limitations in current plant-made therapeutic protein technologies that require regulatory attention and may prevent eventual industry adoption. Potential safety problems exist when plants produce proteins at levels high enough to illicit pharmacological or toxic effects if consumed in the wild by animals or humans. Using edible plant hosts—such as fruits, vegetables, tubers, and nuts—increases the possibility of inadvertently contaminating the food chain. This necessitates the implementation of expensive tracking and sequestering systems when handling such transgenic plants. Other safety concerns are linked to potential genetic drift between plant species, either sexually or otherwise. Horizontal gene transfer between plants, or other wildlife, could also lead to contamination of the food chain and result in potentially harmful species.

One way of mitigating these safety problems and enabling the advantages of plant protein production is to insert intervening polypeptide sequences into the parent therapeutic protein, and thereby disrupt the protein's biological activity. This approach has been demonstrated in plants expressing herbicide resistance proteins by showing that portions of the protein could be fused to inteins, expressed from different parts of the plant genome, and recombined in vivo to produce a fully active protein. The advantage of such a system is in limiting horizontal gene transfer. However, because the fully functional protein is still produced within the plant, this does not eliminate the potential toxicity that could be associated with a plant expressing a therapeutic protein under the same conditions. By breaking a therapeutic protein into multiple segments, each fused to an intein segment that facilitates trans-splicing in vitro, and expressing these fused Intein segments in different plants, the plants can be grown, harvested, and mixed to fully reconstitute active therapeutic proteins in vitro within the manufacturing facility. This approach evades the potential problems associated with producing the fully active proteins in plants in the wild.

### SUMMARY OF THE INVENTION

The present invention provides for genetically recombinant plants, their parts, plantlets, seeds, seedlings, and their progeny (collectively referred to as “plants”), which may contain single or multiple, whole or partial, exogenous gene sequences encoding animal therapeutic proteins, and preferably human therapeutic proteins, each being fused to single or multiple CIVPS or intein sequences, and optionally regulatory sequences suitable for gene expression and transformation of a plant. The modified gene sequences may be expressed constitutively or transiently, throughout the entire plant or in specific tissues, or any combination thereof encompassing both single and multiple intein modified gene sequences. In different embodiments of the invention, any modified gene sequence, or set of modified gene sequences, may be expressed in any or all tissues constitutively or at specific times.

The invention also relates to methods of producing transgenic plants comprising a controllable intervening protein sequence (“CIVPS”) or intein modified genes, e.g. by first constructing a piece of DNA comprising the parent CIVPS or intein modified gene, and transforming the plant with a construct.

The invention also relates to methods of producing a CIVPS or intein modified therapeutic protein, either whole or portions thereof, in transgenic plants, e.g. by transforming the plant, or plant cells, with a single or multiple modified gene sequence(s), and expressing the CIVPS or intein modified protein. Such methods of production extend to two or more therapeutic proteins, either whole or portions thereof, in a transgenic plant or a set of transgenic plants. In one preferred embodiment the gene sequences may be expressed at any time. In another embodiment, prior to the protein(s) being spliced it preferably is (are) provided with a substantially different activity(ies) and/or structural property(ies). The spliced protein product(s) have its(their) activity(ies) unveiled, unless inhibited by an exogeneously added or endogeneously produced molecule(s) analogous to the non-CIVPS or intein modified protein parent sequence. The CIVPS or intein modified gene products may be expressed in large quantities and recovered from the plant material. Alternatively, the plant or plant material may itself be used as a source of CIVPS or intein modified gene products.

The invention also provides for the use of CIVPS or intein modified therapeutic proteins, or intein fused protein portions, expressed in single or multiple plants, the use of single or multiple transgenic plants expressing CIVPS or intein modified genes, either alone or in combination, in batch, semi-batch, and continuous industrial processes for the production of fully active therapeutic proteins.

Other objects, advantages and features of the present invention will become apparent to those skilled in the art from the following brief description of the drawings and discussion.

### BRIEF DESCRIPTION OF THE FIGURES

FIG. 1 is a schematic diagram illustrating the construction of a CIVPS or intein modified therapeutic protein coding DNA sequence constructed by fusion of a CIVPS or intein coding sequence to the coding sequence of a protein of a purported activity, at either the 3′ end of the gene, the 5′ end of the gene, or internally, within the protein gene, and translation of the sequence into a CIWPS or intein modified protein.

FIG. 2 is a schematic diagram illustrating the production of a final active therapeutic protein derived from multiple transgenic plants.

### DETAILED DESCRIPTION OF THE PREFERRED EMBODIMENTS

The present invention is directed to a novel method for the safe production of pharmaceuticals in plants in a cost effective manner by modifying plants through the use of CIVPS or intein modified therapeutic proteins, or protein parts, where the CIVPS or intein is attached to a desired protein portion. The terms “CIVPS” and “intein(s),” as used herein, are intended to refer to similar products, and are used interchangeably. For simplification, the terms “CIVPS” and “intein(s)” are collectively referred to herein as “intervening protein sequence(s).”

Because CIVPS or intein modified proteins, or protein parts, may be expressed in cells at high titer, yet with substantially decreased activity, it has been discovered that, if cloned into single or multiple plants, this decrease in activity would allow the thus formed transgenic plant cells, plant fragments, or plant tissues, to develop into CIVPS or intein modified protein producing complete plants. Moreover, such transgenic plants could be provided in several different embodiments, such as those where the recombinant plants are made to express the modified proteins, or protein parts, 1) constitutively or transiently, 2) through chemical induction or biological induction by the plant's growth cycle, 3) throughout the entire plant or specifically in distinct plant tissues, and/or 4) with or without subcellular localization, among others.

The invention is directed towards the production of therapeutic proteins from transgenic plants, which term as used herein is intended to be synonymous with genetically recombinant plants, their seeds and progeny plants, or any plant portion, tissue or cell, containing a gene(s) for a CIVPS or intein modified therapeutic protein(s) including, but not limited to hormones, growth factors, cytokines, receptors, ligands, antibodies (monoclonal or other), and vaccines. The invention is also directed towards the transgenic plants themselves that contain a gene(s) for a CIVPS or intein modified therapeutic protein(s) including but not limited to hormones, growth factors, cytokines, receptors, ligands, antibodies (monoclonal or other), and vaccines. The invention is further directed towards methods for the production of the transgenic plants that produce CIVPS or intein modified therapeutic proteins, methods for the production of CIVPS or intein modified therapeutic proteins in plants, and uses of the plants as substrates for therapeutic protein production.

Transgenic plants are multi-cellular plants that express single or multiple exogenous genes and their associated protein (or ribonucleic acid) activities. Intervening protein sequences are protein sequences internal or adjacent to a parent protein sequence that may spontaneously cleave themselves at either, or both, the carboxyl or amino terminal ends and are capable of selectively ligating the resulting extein protein fragments when appropriate in either cis or trans reactions, under specific conditions. See, for example, Perler, et al., Nucl. Acids Res., 22:1125-1127 (1994); Wallace, C. J., Protein Sci., 2:697-705 (1993); Xu, et al., Cell, 75: 1371-1377 (1993); Pietrokovski, S., Protein Sci., 2:697-705 (1994). Thus, intervening protein sequences may be said to be in-frame, self-cleaving and potentially self-ligating peptides that generally occur as part of a larger precursor protein molecule. Intervening protein sequences differ from other proteases or zymogens in several fundamental ways. Unlike proteases that cleave themselves or other proteins into multiple, unligated polypeptides, intervening protein sequences have the ability to both cleave and ligate in either cis or trans conformations. Thus, as opposed to terminal cleavage that would result from the reaction of a protease on a protein, intervening protein sequences have the ability to cleave at multiple sites, and ligate the resulting protein fragments. This cleavage may occur spontaneously or may be induced under specific conditions by implementing techniques that are known in molecular biology or biochemistry. Techniques known to induce intervening protein sequence splicing include exposing the intervening protein sequence to a change in temperature (often elevated temperatures are used), decreasing the pH of the solution containing the intervening protein sequence, or exposing the intervening protein sequence to various chemicals, particularly thiol-containing reagents. Furthermore, intervening protein sequence splicing can be inhibited by urea or divalent cations, particularly Zn²⁺. The inhibition from divalent cations can be removed by addition of EDTA. Intervening protein sequences from various sources, their sequences, characteristics and functions have been described fully in the literature. See, for example, Kane et. al., Science 250:651 (1990); Hirata et al., J. Bio. Chem. 265:6726 (1990) (Sacchromyces cerevisiae); Davis et al., J. Bact. 173:5653 (1991), Davis et al., Cell 71:1 (1992) (Mycobacterium tuberculosis); Perler, et al., PNAS 89:5577 (1992) (Thermococcus litoralis).

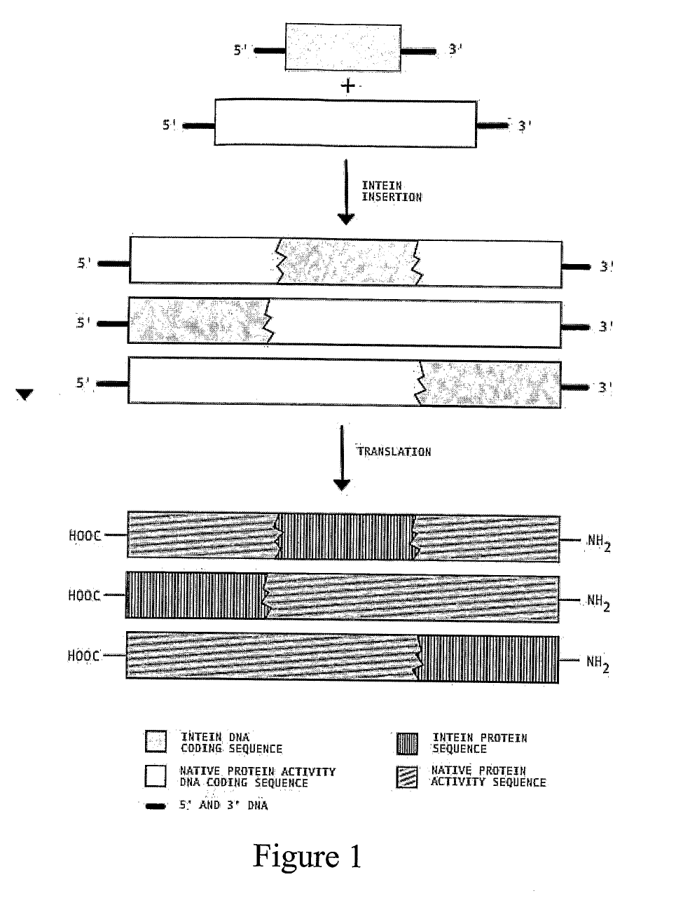

As shown in FIG. 1, the combination of an intein DNA coding sequence with a DNA sequence encoding a therapeutic protein yields an intein modified protein, whose purported activity or structural role may be substantially altered. Transgenic plants that express CIVPS or intein modified proteins (from their associated CIVPS or intein modified genes) are an improvement upon previous transgenic plants, because the parent CIVPS or intein modified protein can have two substantially different states that are controllably mediated by intervening protein sequence cleavage. This cleavage may or may not be associated with recombination of the purported protein sequence.

The invention may be formed from any single or multiple plant species, combined with any combination of single or multiple proteins and one or more intervening protein sequences. Plant species may include, but are not limited to: poplar, birch, cedar, pine, hardwoods, softwoods, soybeans, switchgrass, corn, tobacco, alfalfa, sugar cane, cauliflowers, artichokes, bananas, apples, cherries, cranberries, cucumbers, lettuce, grapes, lemons, melons, nuts, tangerines, rice, oranges, peaches, pears, blueberries, strawberries, tomatoes, carrots, cabbages, potatoes, endive, leeks, spinach, weeds, arrowroot, beets, carrots, cassaya, turnips, yams, radishes, sweet potatoes, wheat, barley, soya, beans, rapeseed, millet, sunflower, oats, peas, tubers, bamboo, seaweed, algae, or any other plant species.

Proteins may include any known, putative, modified, or de novo created proteins of therapeutic value. Although the selection of the native protein is not restricted, preferred proteins include hormones (insulin, growth hormone, antiduretic hormone), growth factors (erthyropoietin, epidermal growth factor, insulin-like growth factor, fibroblast growth factor), cytokines (IL-2, IL-6), enzymes (trypsin, gastric lipase, glucocerebrosidase, urokinase, iduronidase), bacterial or viral antigens, antibodies, receptors, and other therapeutic proteins implicated in disease pathogenesis and all of their associated isoforms.

The choice of intervening protein sequence(s) used to modify the protein, the fusion of which is expressed in the desired plant, is also not limited. Any single or multiple intervening protein sequence may be used in any configuration with respect to the desired protein or proteins. The intervening protein sequence should have the capability to be spliced at one or both ends in response to some stimuli, and may or may not permit ligation of the proteins to which single or multiple intervening protein sequences are fused.

Transgenic plants expressing CIVPS or intein modified proteins, and the production of CIVPS or intein modified proteins in transgenic plants can be accomplished by constructing a DNA sequence containing the CIVPS or intein modified protein of interest and the necessary regulatory elements required for its expression, amplification and selection of the constructed DNA, transformation of the desired plant species, regeneration and selection of the appropriately transformed plant species, and if necessary, purification of the CIVPS or intein modified protein in its native form or the cleaved form. Both the production of transgenic plants expressing CIVPS or intein modified therapeutic proteins, and the production of CIVPS or intein modified therapeutic proteins in transgenic plants form part of this invention.

For the production of the transgenic plants, or CIVPS or intein modified proteins in transgenic plants, the CIVPS or intein modified protein DNA sequence must be constructed. This can be accomplished by cloning the entire gene sequence, or portions thereof, of the desired therapeutic protein and the desired intervening protein sequence into E. coli or any other suitable host (e.g., yeast may be beneficial in some cases, or expression in mammalian or plant cells with or without the use of viral or non-viral vectors).

Once the gene and sequence encoding the intervening protein sequence have been cloned, they are joined in the desired configuration. The chosen intervening protein sequence should be able to perform the desired functions such as spontaneous splicing or splicing in response to an imposed stimuli (for example, light, pH change, temperature, pressure, or changes in the local chemical composition surrounding the CIVPS or intein modified protein), and if necessary permitting ligation of the fused protein either with itself in cis, or with another protein in trans. Joining the intervening protein sequence's DNA sequence and the protein's DNA sequence is easily accomplished by direct polynucleotide synthesis or methods known in the art, resulting in CIVPS or intein modified protein DNA coding sequences, or combinations thereof, as shown in FIG. 1. As already indicated, a CIVPS or intein modified protein is one which fuses the intervening protein sequence to one of either the carboxy terminal, amino terminal, or internal portions of the native protein or proteins. Although many alternative methods exist, one way of creating the fusion between the sequences encoding the intervening protein sequences and the desired protein coding sequences would be to synthesize or purify the DNA encoding the desired therapeutic protein sequence, use a restriction enzyme to cut the protein coding sequence at the desired point of CIVPS or intein insertion, and then ligate the CIVPS or intein coding sequence into the restricted site. Another method is to use PCR to combine the genetic elements.

The polynucleotide, or either of the nucleic acid segments, may be cloned directly to appropriate regulatory and/or selection sequences, or via a vector. Examples of regulatory segments are promoters to control the temporal expression of the CIVPS or intein modified protein, origins of replication, and/or signaling sequences to control the spatial distribution of CIVPS or intein modified proteins in vivo in specific plant tissues and/or specific subcellular compartments. Examples of selection elements include herbicidal or antibacterial genes, fluorescent markers, dye markers, and other suitable selective markers. The resulting polynucleotide or vector comprising the CIVPS or intein modified protein(s) encoding polynucleotide(s), and optionally any desired regulatory and selection elements, may then be amplified to obtain larger amounts of product, which may be used for subsequent transformation of a desired plant species.

Modification of any and all of these steps is possible to facilitate specific orientation and fusion between any desired intervening protein sequence(s) and protein(s). Alteration of either the protein's coding sequence and/or the intervening protein sequence's coding sequence and the ligation of either or both of these sequences may be accomplished by techniques known in the art, such as site-directed mutagenesis, computational mutagenesis and selection, random mutagenesis, polymerase chain reaction (PCR), error-prone PCR, and/or any other suitable method that would be considered routine by an artisan. These techniques facilitate the placement of a number of joining sequences, and any desirable and suitable combination may be used. Likewise, any combination or orientation of regulatory and selective elements may also be implemented in accordance with this invention.

Gene regulatory elements, such as promoters (Guilley et al., Higgins, T. J. V., Coruzzi et al., Tingey et al., Ryan et al., Rocha-Sosa et al., Wenzler et al., Bird et al.), enhancers (Brederode, et al.), RNA splicing sites, ribosomal binding sites, glycosylation sites, protein splicing sites, subcellular signaling sequences (Smeekens et al., van den Broeck et al., Schreier et al., Tague et al.), secretory signal sequences (Von Heijne, G., Sijmons, et al.), or others may be advantageous in controlling either the temporal or spatial distribution, extent and form or glycosylation, or structure of the CIVPS or intein modified protein concentration and activity in vivo in the transformed plant or subsequent therapeutic activity in human or non-human animals. Use of these elements may be desired to facilitate the production and processing of CIVPS or intein modified proteins from transgenic plants into therapeutic proteins. The expression of the CIVPS or intein modified protein(s) may be conducted either in a constitutive or induced manner. In order to attain either of these modes, any of the methods that are either described herein or known in the art, or later made available, may be implemented. The induction of protein expression may be attained with the aid of one or more foreign stimuli. Examples include the exposure to a pesticide(s), to light, a temperature change(s), and/or sound(s), however, other foreign stimuli may also be employed. In addition, the recombinant plant may also express any one or more of the selectable marker gene or reporter gene(s) that provide the plant with resistance to chemicals including bromoxynil, 2,2-dichloropropionic acid, G418, glyphosphate, haloxyfop, hygromycin, imidazoline, kanamycin, methotrexate, neomycin, phosphinothricin, sethoxydim, 2,2-dichloropropionic acid, trichothecne, sulfonylurea, s-triazine, and/or triazolopyrimidine.

Once the CIVPS or intein modified protein DNA sequence has been constructed, combined with the desired regulatory and selection DNA sequences, successfully cloned and selected, then the transformation of the desired plant species and generation of full plants is required. Methods for the transformation of a desired plant species, and the generation of full plants, can be accomplished by techniques known in the art (Draper, et al., Potrykus, et al., Broothaerts, et al.). Transformation techniques include, but are not limited to: Agrobacterium tumefaciens mediated gene transfer, Agrobacterium rhizogenes mediated gene transfer, Sinorhizobium meliloti mediated gene transfer, Rhizobium mediated gene transfer, Mesorhizobium loti mediated gene transfer, direct gene transfer to plant protoplasts, Ti plasmid mediated gene transfer (with or without a helper plasmid), biolistic or particle bombardment plant transformation (Gordon-Kamm et al.), microinjection and fiber-mediated transformation, viral transformation, and tissue electroploration (Shimamoto et al.). Gene transfer may occur in whole plants, plant explants (such as, but not limited to root explants), any plant portion (such as, but not limited to plant leaf segments, seeds, or seed segments), plant protoplasts or apoplasts, or single or multiple plant cells.

Methods of selection of properly transformed plants are also known in the art. Selection methods may be facilitated by including a selectable marker in the transformed DNA containing the CIVPS or intein modified protein (such as a resistance gene, gene coding the production of a colored compound, gene coding the production of a fluorescent compound, or any other suitable method). Additionally, DNA from transformed plants may be isolated and analyzed to confirm the presence of the desired CIVPS or intein modified protein coding sequence. Techniques that are suitable for confirmation of the selection process include DNA sequencing, polymerase chain reaction, restriction digest analysis and southern analysis. Any method of selection that allows identification of the desired transgenic plant may be used.

Once the plant is transformed with the CIVPS or intein modified protein and desired regulatory and selection sequences, whole plants can be regenerated by methods know to the art (Horsch et al.). Most methods consist of culturing the transformed plant cells, explants, tissues, parts, or whole plants in the proper medium and under appropriate conditions of light and temperature. The method used to regenerate the plant should not limit the invention and any effective method may be used.

Once the whole, transgenic plant has been selected, it can be monitored for CIVPS or intein modified protein expression. This is not required for the production of transgenic plants expressing CIVPS or intein modified proteins, but it is prudent to confirm that the desired transgenic plant expressing the desired CIVPS or intein modified protein has been obtained and expression is properly controlled by the desired control elements used. Protein expression of the CIVPS or intein modified protein can be monitored by western analysis, radio-immuno assay (RIA), in situ hybridization, 2-dimensional gel electrophoresis (and staining), or mass spectrometry, conducted on plant extracts or protein fractions purified from the transgenic plant. In addition, either some of the purified proteins, or the transgenic plant itself, should be exposed to conditions that permit CIVPS or intein cleavage. After exposure, both the CIVPS or intein modified protein and the resulting protein that appears as a consequence of CIVPS or intein cleavage can both be analyzed by western analysis, and other assays, to verify the presence of the appropriate proteins, and the difference in activity between the CIVPS or intein modified protein and the resulting cleaved protein. The activity assays should be designed so as to monitor the desired protein activity and should be specific to that activity and not vulnerable to competing interferences. A control can be used as a standard to compare the native activity with both the CIVPS or intein modified activity and the activity following CIVPS or intein cleavage.

Methods and processes using transgenic plants expressing CIVPS or intein modified proteins include the use of the plants as substrates for therapeutic protein production, and the use of the plants for vaccine delivery. Any batch, semi-batch, or continuous process in which transgenic plants that express CIVPS or intein modified proteins are used as substrates for one of the purposes described above is within the spirit of this invention. These processes may include, but are not limited in scope to, processes in which the transgenic plants expressing CIVPS or intein modified proteins are harvested, exerted to the CIVPS or intein cleavage stimuli, mixed with other substrates in a substrate to transgenic plant ratio greater than or equal to zero, and then converted either chemically, enzymatically, or biologically to one of the products detailed above.

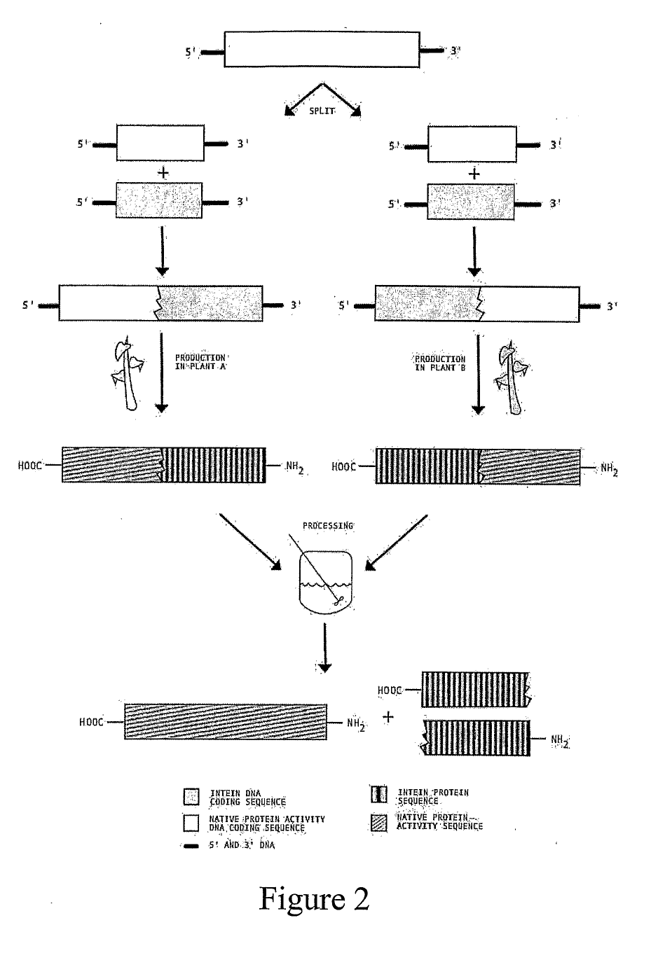

The present invention is also directed to a process for making therapeutic proteins from plants expressing CIVPS or intein modified proteins as shown in FIG. 2. The process comprises dividing the nucleic acid coding sequence of a therapeutic protein into multiple segments; fusing each segment in-frame with a sequence encoding an intervening protein sequence capable of splicing in trans; constructing an expression vector that allows for the expression of the intervening protein fused coding sequence and selection of plants transformed with the vector; genetically modifying multiple plants (either sexually compatible or incompatible) with each of the expression vectors previously formed such that the entire parent coding sequence of the therapeutic protein is contained within the set of plants; growing the transgenic plants; harvesting the transgenic plants; milling the transgenic plants and combining them in a liquid manufacturing process whereby the intervening protein sequence fused protein portions are exposed to conditions that permit splicing and reforming the parent therapeutic protein, followed by purification of the protein.

In one embodiment of this invention, the expressed CIVPS or intein modified protein(s) is(are) comprised of a parent protein sequence(s), whose activity(ies) may be known, inferred through sequence or structure homology and/or produced by mutagenesis or by de novo synthesis. Each parent sequence(s) is divided into subsequences and fused to, an intervening protein sequence(s). Once inserted, the modified protein(s) expressed from the fused subsequences are inactive, in vivo. This embodiment can be extended to two or more transgenic proteins in one plant or a set of plants. The complete parent protein's original activity may be substantially recovered, if and when desired, by CIVPS or intein splicing either in cis or in trans when contacted by other CIVPS or intein modified proteins thus designed and in the presence of appropriate splicing stimulus. For example, in one application, following plant harvest and during mixing of two plants, each expressing a portion of a therapeutic protein fused to an intervening protein sequence, each intervening protein sequence may be induced to splice itself from its parent protein sequence, joining the two therapeutic protein portions and which parent protein now has recovered its original or intended activity, affinity, or ability to act as a ligand or hormone. Methods for CIVPS or intein splicing with, or without, recombining of the protein to a functioning activity are known to one skilled in the art, and need not be repeated here. These methods include the use of light, temperature, change in pH, and/or the addition of chemical reagents.

FIG. 2 illustrates the production of a final active therapeutic protein derived from multiple transgenic plants. In the first step, the entire DNA coding sequence of the desired therapeutic protein is divided into two or more pieces. Each piece is then fused with an intein protein coding sequence. The fusion construct is then packaged into an appropriate expression vector and each piece of the therapeutic protein, fused with an intein that is capable of splicing in trans, in vitro, is used to transform a plant, which subsequently expresses the construct. The plants are then harvested, and the proteins are extracted and mixed, which allows the protein fusions to splice in trans, removing the intein portions and relegating the resulting therapeutic protein portions such that the fully active therapeutic protein is recovered. Cleavage of the CIVPS or intein modified protein(s), or components thereof, may be attained in vitro when subjected to an appropriate cleavage environment. While FIG. 2 schematically illustrates an example of the entire process for production of a therapeutic protein, one of ordinary skill in the art would appreciate that other variants may be constructed as combinations of the CIVPS or intein modified proteins.

The present invention is also directed to recombinant plants, or plant parts, plantlets, tissues, cells, sub-cellular fractions, seeds, seedlings, protoplasts, progeny or descendents, comprising an expression construct(s) that encode(s) at least one modified protein comprising a target protein(s) or protein segment(s), which is(are) fused, either internally or terminally, to a CIVPS or intein sequence(s) or segment(s) thereof, or to an amino terminus(i) or a carboxyl terminus(i) thereof. In one embodiment, each expression construct of the plants, or plant parts, plantlets, tissues, cells, sub-cellular fractions, seeds, seedlings, protoplasts, progeny or descendents comprises, operatively linked to one another, a first sequence of nucleic acids encoding a target protein or proteins, and a second sequence of nucleic acids encoding a CIVPS or intein sequence or sequences, and optionally selectable markers or reporter genes and/or promoters. It is understood that in a more specific embodiment the sequences may be fused, either directly or via one or more linkers, and more preferably in reading frame. The modified protein or proteins may be expressed by the plants, or plant parts, plantlets, tissues, cells, sub-cellular fractions, seeds, seedlings, protoplasts, progeny or descendents either constitutively, or inductively. In the latter case, the expression and/or splicing of the at least one modified protein may be triggered or induced by one or more stimuli. Examples of suitable stimuli comprise a pH change, change in osmolality, or temperature, the addition of a chemical, or a change in light, and/or sound.

The plants, or plant parts, plantlets, tissues, cells, sub-cellular fractions, seeds, seedlings, protoplasts, progeny or descendents may express the modified proteins either at a pre-determined point of the plant life cycle, in one or more specific tissues or parts thereof, and/or in at least one specific sub-cellular compartment. Alternatively or in conjunction with the latter, the modified proteins may be expressed and secreted extracellularly. The plants, or plant parts, plantlets, tissues, cells, sub-cellular fractions, seeds, seedlings, protoplasts, progeny or descendents specific tissue may be seeds, roots, fruits, stems, tubers and/or leaves, and the specific subcellular compartments may be a cellular cytosol, apoplast, mitochondrion, plastid, endoplasmic reticulum, inclusion body, vacuole and/or nucleus. Other variations, however, are also included within the confines of this invention.

The plants, or plant parts, plantlets, tissues, cells, sub-cellular fractions, seeds, seedlings, protoplasts, progeny or descendents may also carry a selectable marker that confers it resistance to a chemical. Examples of selectable markers include bromoxynil, 2,2-dichloropropionic acid, G418, glyphosphate, haloxyfop, hygromycin, imidazoline, kanamycin, methotrexate, neomycin, phosphinothricin, sethoxydim, 2,2-dichloropropionic acid, trichothecne, sulfonylurea, s-triazine, and/or triazolopyrimidine. Others, however, may also be employed. The promoter may be included to precede a CIVPS or intein-modified protein polynucleotide. In some cases, the plants, or plant parts, plantlets, tissues, cells, sub-cellular fractions, seeds, seedlings, protoplasts, progeny or descendents may be tolerant or resistant to normally extremely toxic levels of a selected chemical or chemicals.

In another embodiment, the plants, or plant parts, plantlets, tissues, cells, subcellular fractions, seeds, seedlings, protoplasts, progeny or descendents are fertile, and has at least one heritable modified protein encoding polynucleotide sequence. However, it may just as well not be fertile. Further, as indicated above, this invention extends to inbred and hybrid genetically recombinant plants, or plant parts, plantlets, tissues, cells, sub-cellular fractions, seeds, seedlings, protoplasts, progeny and descendents, which may or may not be produced by the method of this invention. Of particular interest are plant parts, plant seeds, plant seedlings and plant protoplasts, which have substantial commercial importance. Also of commercial and other interest are plants, plant tissues, plant cells, and sub-cellular fractions.

In one aspect, the intervening protein sequence and the target protein or protein segment form at least one splice junction with the target protein. In a desirable embodiment, the amino acid residue at the carboxyl terminus(i) of the splice junction(s) is(are) provided with a hydroxyl or a sulfhydryl side chain(s). In another particularly useful embodiment, the splice junction(s) can be located downstream of the intervening protein sequence(s) or segment(s) thereof, and may comprise(s) an amino acid residue(s) lacking, for example, hydroxyl or sulfhydryl side chains at the amino terminus(i) of the target protein or protein segment(s). In another variation, the splice junction(s) can be located upstream of the intervening protein sequence(s) or segment(s) thereof, and may comprise an amino acid residue(s) having hydroxyl or sulfhydryl side chains at the amino terminus(i) of the intervening protein sequence(s) or segment(s) thereof. In addition, the splice junction(s) can be located upstream of the intervening protein sequence(s) or segment(s) thereof, and may comprise a cysteine. Still another important variation is that the splice junction(s) can be located downstream of the intervening protein sequence(s) or segment(s) thereof, and may be provided with His-Asn at the carboxyl terminus(i) of the intervening protein sequence(s) or segment(s) thereof, and/or with an amino acid residue(s) having hydroxyl or sulfhydryl side chains at the amino terminus(i) of the adjoining region(s) of the target protein(s). In yet another variant, the splice junction(s) can be located downstream of the intervening protein sequence(s) or segment(s) thereof, and may be provided with an Asp, Asn, Glu, or Gln at the carboxyl terminus(i) of the intervening protein sequence(s) or segment(s) thereof, and/or with an amino acid residue(s) having hydroxyl or sulfhydryl side chains at the amino terminus(i) of the adjoining region(s) of the target protein(s) or protein segment(s).

Further modifications include those where the Asp at the carboxyl terminus(i) is replaced by an amino acid(s) lacking carboxyl or amino side chains, and where the intervening protein sequence(s) or its segment(s) comprise(s) an externally controllable intervening protein sequence(s) or segment(s) thereof. Other constructs suitable for insertion in the products of the invention are those where the intervening protein sequence(s) or segment(s) thereof is(are) inserted immediately before Ser, Thr or Cys of the target protein(s) or protein segment(s), and where the intervening protein sequence(s) amino or carboxy terminus(s) comprise(s) Ser, Thr or Cys, among others.

As described in more detail below, the therapeutic protein, or protein portions, may be expressed in human or non-human animals, viruses, or microorganisms, such as a bacterium, as is known in the art. Preferred target proteins include insulin, erythropoietin, growth hormone, epidermal growth factor, serum albumin, trypsin, insulin-like growth hormone, tumor necrosis factor, tumor necrosis factor receptor, her2 receptor, monoclonal antibodies, and other hormones or growth factors, and all of their respective isoforms.

Another embodiment of the present invention includes the expression of the modified protein by a virus. Although any virus could be employed, examples are HIV, hepatitis, SARS, human pappiloma virus, influenza, rotavirus, and varicella, among others.

The recombinant plants, or plant parts, plantlets, tissues, cells, sub-cellular fractions, seeds, seedlings, protoplasts, progeny or descendents may be produced by a method comprising providing an expression construct that encode(s) at least one modified protein comprising a target protein, or protein segment(s), which is(are) fused, either internally or terminally, to a CIVPS or intein sequence(s) or segment(s) thereof, or to an amino terminus(i) or a carboxyl terminus(i) thereof; transforming multiple plants, or plant parts, plantlets, tissues, cells, sub-cellular fractions, seeds, seedlings, protoplasts, progeny or descendents, with an expression construct; and regenerating a genetically recombinant plants, or plant parts, plantlets, tissues, cells, sub-cellular fractions, seeds, seedlings, protoplasts, progeny or descendents, from the transformed plants, or plant parts, plantlets, tissues, cells, sub-cellular fractions, seeds, seedlings, protoplasts, progeny or descendents, that encode(s) at least one modified protein sequence(s).

It is highly preferred that transformation be stable. However, transformations that have some temporary stability are also desirable. The regeneration step may be conducted by breeding of the recombinant plants, or plant parts, plantlets, tissues, cells, sub-cellular fractions, seeds, seedlings, protoplasts, progeny or descendents; crossing of a recombinant plants, or plant parts, plantlets, tissues, cells, sub-cellular fractions, seeds, seedling, protoplasts, progeny or descendents and a non-genetically recombinant plant, or plant part, plantlet, tissue, cell, sub-cellular fraction, seed, seedling, protoplast, progeny or descendent; and/or back-crossing of two genetically recombinant plants, or plant parts, plantlets, tissues, cells, sub-cellular fractions, seeds, seedlings, protoplasts, progeny or descendents.

The expression construct employed in this method may comprise one or more of a promoter, selectable marker, resistance marker, heritable marker, poly-adenylation sequence, repressor, enhancer, localization sequence, and/or signaling sequence. In an important aspect of the method, the plants, or plant parts, plantlets, tissues, cells, sub-cellular fractions, seeds, seedlings, protoplasts, progeny or descendents are transformed with the expression construct by either viral transformation, bombardment with DNA-coated microprojectiles, liposomal gene transformation, bacterial gene transfer, electroporation, or chemical gene transformation, or more than one of these. As indicated above, the plants, or plant parts, plantlets, tissues, cells, sub-cellular fractions, seeds, seedlings, protoplasts, progeny or descendents, may be transformed by means of a bacterium, e.g. Agrobacterium tumefaciens or Sinorhizobium meliloti, although other microorganisms may also be employed. In the present method, the transformation may be conducted by chemical gene transformation, and it may be done with the aid of, e.g. calcium phosphate, and/or polyethylene glycol, or other chemicals known in the art as being suitable for this purpose. The selection may be attained with the aid of a selectable marker, or a resistance marker, or of the expression of at least one nucleic acid encoding a CIVPS or intein modified protein. In the method of the invention, the genetically recombinant plants, or plant parts, plantlets, tissues, cells, sub-cellular fractions, seeds, seedlings, protoplasts, progeny or descendents may be regenerated from a transformed embryogenic tissue(s); plant protoplasts; cells derived from immature embryos; or from transformed seeds, among other sources.

The present invention also provides a method for producing a modified protein(s) or protein segment(s), as well as fully active reformed therapeutic proteins, from a single or multiple recombinant transformed plant(s), or plant part(s), plantlet(s), tissue(s), cell(s), sub-cellular fraction(s), seed(s), seedling(s), protoplast(s), progeny or descendent(s) expressing the protein(s) or protein segment(s), that comprises conducting the method described above, and further harvesting the modified protein(s) or protein segment(s) from the transformed plants, or plant parts, plantlets, tissues, cells, sub-cellular fractions, seeds, seedlings, protoplasts, progeny or descendents. The method may further comprise reforming the proteins in vitro by mixing multiple plants expressing different subsequences of the parent protein, each fused to an intervening protein sequence, and using CIVPS or intein splicing to reform the original parent sequence after harvesting. The method may further comprise purifying the modified protein(s). As described here, this method may produce a modified protein(s) or protein segment(s) that comprises a CIVPS or intein modified protein(s) or protein segment(s) or a fully mature protein lacking the previously fused intervening protein sequence after splicing.

The present invention also provides a method for producing a modified protein comprising a target protein(s) or protein segment(s) fused, either internally or terminally, to an intervening protein sequence(s) or segment(s) thereof, or to its amino terminus(i) or carboxyl terminus(i). The method comprises obtaining an expression construct encoding a target protein having an in-frame fused intervening protein sequence(s) or segment(s) thereof, or its amino terminus(i) or carboxyl terminus(i); transforming a host plant cell(s) with the expression construct; and culturing the transformed plant host cell under conditions effective for expressing the modified protein.

In one preferred aspect, in the expression construct, at least one first nucleic acid segment(s) encoding the intervening protein sequence(s) or segment(s) thereof is(are) fused to the 5′-end of a second nucleic acid segment(s) encoding the target protein(s) or protein segment(s). Alternatively, in the expression construct the first nucleic acid segment(s) encoding the intervening protein sequence(s) or segment(s) thereof may be fused to the 3′-end of the second nucleic acid segment(s) encoding the target protein(s) or protein segment(s). It is particularly suitable to practice the present method to employ an intervening protein sequence(s) or segment(s) thereof, which is known to effect, either in cis or in trans, excision, cleavage, ligation, excision-ligation, cleavage-ligation, and/or cyclization. When the intervening protein sequence(s) or its(their) segment(s) are employed to induce protein splicing, this event may be induced or triggered by a change of temperature, light or pH, the addition/removal of a chemical reagent that facilitates/inhibits splicing or cleavage, amino acid dephosphorylation or deglycosylation, or by contact with, or removal of, a peptide or peptidomimetic activating or blocking of splicing or of cleavage.

Another manner of inducing protein splicing is either in vitro or in vivo contact with, or removal of, a peptide or peptidomimetic agent that may either activate or block splicing or cleavage. Interesting variations that produce superior results are those where the amino or carboxy terminus(i) of the intervening protein sequence(s) or segment(s) thereof comprise(s) Ser, Thr or Cys, or where the carboxyl terminus(i) of the intervening protein sequence(s) or segment(s) thereof comprise(s) on of Asp, Asn, Gln, or Glu preceding Ser, Thr or Cys of the target protein(s) or protein segment(s). However, other modifications are also possible, as is known in the art.

In the present method, the expression construct may further comprise a promoter, a selectable marker, a resistance marker, a heritable marker, a poly-adenylation sequence, a repressor, an enhancer, a localization sequence, or a signaling sequence. Moreover, the method presented here may also comprise the transformation of the plants, or plant parts, plantlets, tissues, cells, sub-cellular fractions, seeds, seedlings, protoplasts, progeny or descendents with the expression construct being implemented by viral transformation, bombardment with DNA-coated microprojectiles, liposomal gene transfer, bacterial gene transfer, electroporation, and/or chemical gene transformation, and/or other methods known in the art, or that will be subsequently developed. As described above, in the method described here, the bacterium used to transfer the expression construct may be an Agrobacterium tumefaciens or other bacterium; the chemical used for transformation may be calcium phosphate, or polyethylene glycol; the transformed plant cells, plant parts, plants, etc. may be selected through their expression of a selectable marker, or resistance marker; the selection of the transformed plant, or plant part, plantlet, tissue, cell, sub-cellular fraction, seed, seedling, protoplast, progeny or descendent may be conducted through their expression of the modified protein gene sequence; and the regeneration of the genetically recombinant plant, or plant part, plantlet, tissue, cell, sub-cellular fraction, seed, seedling, protoplast, progeny or descendent may be attained from transformed embryogenic tissue; from cells derived from immature embryos; or from transformed seeds, among others.

The present invention is also directed to a method for producing seed(s) that express one or more modified proteins. The method comprises obtaining the genetically recombinant plant, or plant part, plantlet, tissue, cell, sub-cellular fraction, seed, seedling, protoplast, progeny or descendent of the invention; culturing or cultivating the genetically recombinant plant, or plant part, plantlet, tissue, cell, sub-cellular fraction, seed, seedling, protoplast, progeny or descendent; and obtaining from the cultivated plant seed that expresses one or more modified proteins.

Another method provided by the present invention is one for using one or more plant(s), or plant part(s), plantlet(s), tissue(s), cell(s), sub-cellular fraction(s), seed(s), seedling(s), protoplast(s), progeny or descendent(s) expressing one or more modified proteins for producing a therapeutic protein. The methods comprises harvesting one or more recombinant plants, or plant part(s), plantlet(s), tissue(s), cell(s), sub-cellular fraction (s), seed(s), seedling(s), protoplast(s), progeny or descendent(s) in accordance with the teachings of the present invention; mechanically processing the plant(s), or plant part(s), plantlet(s), tissue(s), cell(s), sub-cellular fraction(s), seed(s), seedling(s), protoplast(s), progeny or descendent(s); combining the mechanically processed plant(s), or plant part(s), plantlet(s), tissue(s), cell(s), sub-cellular fraction(s), seed(s), seedling(s), protoplast(s), progeny or descendent(s), with other genetically recombinant plants in a proportion greater than or equal to zero; and chemically processing the plants or specific portions of the plants under conditions effective for obtaining the chemical compound. The chemical compound may be a therapeutic protein, or other chemical of interest produced from the parent protein, which was produced using this method.

This method may be practiced by mechanical processing of one or more plants, or plant part(s), plantlet(s), tissue(s), cell(s), sub-cellular fraction(s), seed(s), seedling(s), protoplast(s), progeny or descendent(s) by extrusion, grinding, shredding, mulching, chipping, dicing, compressing, exploding, and/or tearing. Other processing techniques, however, are also suitable. The chemical processing of the combined components may be attained by various techniques or a combination thereof. Some of them are pre-treatment with steam, dilute or concentrated acid, ammonia explosion, sterilization, soaking in water, mixing with a solvent, a change of pH, temperature or osmolality, exposure to or changes in light, inorganic and/or enzyme catalysis, saccharification, bleaching, scouring, fermentation, distillation, chromatography, adsorption, and/or addition of a chemical(s). Others, of course, are also employed successfully. Various steps are of use when practiced as follows: the pre-treatment may include soaking the combined products for extraction purposes; the chemical processing may be attained by pre-treatment with at least one of sulfuric acid, hydrochloric acid, phosphoric acid, or carbonic acid, sodium hydroxide, organic or inorganic base, or by soaking in water at a temperature greater than or equal to about 20° C., and/or by mixing the combined products with at least one of water, or an organic or inorganic solvent(s). As already explained, an external stimulus(i) may be applied to induce splicing of the modified protein(s) or protein segment(s). Examples of external stimuli are a change of pH, osmolality, or temperature, exposure to sound, light, or addition of a chemical(s).

In some cases the spliced proteins or protein segments may exhibit altered activities with respect to the modified proteins or protein segments, such as altered catabolic, anabolic, affinity, binding or therapeutic activities with respect to the original target proteins. Examples of spliced proteins or protein segments are those derived from erthyropoietin, insulin, growth hormone, tumor necrosis factor, tumor necrosis factor receptor, her2 receptor, epidermal growth factor, fibroblast growth factor, insulin-like growth hormone, angiotensin, factor V, factor VII, antimicrobial peptides, antibodies, or other hormones or growth factors, and all of their associated isoforms. Thus, the spliced protein may be capable of producing the mature therapeutic protein after splicing and ligation.

A further aspect of this invention involves a method for producing one or more target proteins or protein segments. The method comprises producing a first modified protein (or protein segment, wherein the amino terminus of an intervening protein sequence or segment thereof is fused to the carboxyl terminus of a target protein or protein segment by the method or methods described above; producing a second or more modified proteins comprising a segment of the intervening protein sequence; contacting the first and second or more modified proteins under conditions effective for trans cleavage of the intervening protein sequence or segment thereof by the second modified protein; and repeating this process until the target protein is fully reformed with the desired activity.

Yet another variation of the above method for producing one or more target proteins comprises producing a first modified protein, wherein the carboxyl terminus of an intervening protein sequence or segment thereof is fused to the amino terminus of the target protein or protein segment by the already described method; similarly producing a second or more modified proteins or protein segments comprising a segment of the intervening protein sequence; and contacting first and second or more modified proteins under conditions effective for trans cleaving the intervening protein sequence or segment thereof from the first modified protein or protein segment, and repeating this process with all protein parts until the final desired target protein is obtained. The cleavage may be induced in this procedure by a change in temperature, light, or pH, addition/removal of chemical that facilitates/inhibits splicing or blocking of cleavage, amino acid dephosphorylation or deglycosylation, and/or contact/removal of peptide or peptidomimetic that activates/blocks splicing/cleavage, among others.

It should also be noted that the use of the present invention is not limited to manufacturing processes or mechanical processes. Non-limiting examples of applications of this invention are in the delivery of vaccines, hormones, or therapeutic proteins, in which case the CIVPS or intein modified protein may comprise a combination of therapeutic protein(s) and/or protein antigen(s), potentially protective protein sequences, and intervening protein sequence(s) that may be expressed by the transgenic plant, e.g. a banana, or soy bean plant. The delivery process may occur, for example, by ingestion of the plant product by a human or non-human animal. The plant is then masticated in the mouth and exposed to a stimulus(i) in vivo in the stomach, which in turn triggers or induces cleavage by the intervening protein sequence. In the case of humans, the stimulus may be the reduced pH of the stomach, which induces the cleavage of the intervening protein sequence from the antigen or therapeutic protein, and provides for appropriate ligation, if necessary. The therapeutic protein or antigen would then flow into the duodenum, or small intestine, where the pH would be neutralized and protein products are now ready to be absorbed into the blood stream.

### EXAMPLES

### Example 1

### Production of Human Growth Hormone from Multiple Tobacco Plants using Intein Modification

The activity and structure of human Growth Hormone (hGH, also called human somatotropin) has been studied in detail and several forms are currently licensed for therapeutic intervention in growth hormone deficiency, Turner Syndrome, chronic renal failure, and HIV wasting syndrome. The native form of human somatotropin has been previously expressed in plants (Staub, et. al, 2000).

This example describes the procedure for producing human Growth Hormone (hGH, also called human somatotropin) from intein-modified hGH genes in tobacco plants. In this example, we have selected the codon optimized version of the hGH gene (GenBank Accession # AF205361) [SEQ ID NO: 1] and the split intein from Synechocystis Sp. (GenBank Accession # AF545504 (In), AF54505 (Ic)) [SEQ ID NOS: 2 and 3]. Although variations of the different steps can be used to practice this invention, the procedure proceeds by: 1) creating intein modified hGH genes, 2) packaging the genes into appropriate tobacco expression vectors, 3) transforming tobacco with the expression vectors, and 4) selecting and regenerating fully developed tobacco plants that express the intein-modified genes. From these plants the intein-modified proteins can be recovered and mixed to induce splicing and reconstitute actively formed hGH (as shown in FIG. 2).

Design of the intein-modified hGH-In and Ic-hGH fusion genes requires either splitting the hGH gene immediately before a native serine, threonine or cysteine codon, or adding an artificial codon for insertion anywhere within the gene. We have selected the native serine site at basepair 241 of the hGH gene [SEQ ID NO: 1] to make a break. The hGH-In gene was then synthesized (Blue Heron, Bothell, Wash.) resulting in a DNA sequence encompassing the start codon of hGH (basepair 1) to basepair 240, connected directly to the dnaE amino intein (In) sequence from Synechocystis (GenBank # AF545504) [SEQ ID NO: 2]. Similarly, the Ic-hGH gene was synthesized using the coding sequence for the dnaE carboxy intein (Ic) from Synechocystis (GenBank # AF54505) [SEQ ID NO: 3] followed directly by basepair 241 of the hGH gene and extending all the way to the gene terminus (basepair 582). These fusion products can also be readily constructed using PCR and other molecular biology techniques.

Once synthesized, the hGH-In gene and the Ic-hGH gene are cloned into the Agrobacterium tumefaciens binary expression vector pBI121 (GenBank Accession # AY781296) [SEQ ID NO: 4] as described previously (Chin et. al. 2003) to create pBIhGHIn and pBIIchGH, respectively. A. tumefaciens strain LBA4404 was then electroporated with each vector to yield two different sets of colonies: one set containing pBIhGHIn and one set containing pBIIchGH. A colony from each set was then picked and grown in LB at 30° C. and 200 rpm overnight. The following morning, cultures were diluted 1:1 in LB and allowed to grow to an A₅₅₀ of approximately 1.0. For transformation, leaf sections from 28 to 35 day old tobacco shoot cultures can be used. One group of leaf sections are incubated with the A. tumefaciens containing pBIhGHIn and another group of leaf sections are incubated with A. tumefaciens containing pBIIchGH. The incubation occurs for 2-3 days at 28° C. in transformation medium (1× MS salts, 3% sucrose, 2 mg/L α-napthaleneacetic acid, 0.5 mg/L benzylaminopurine). Leaf sections are transferred to selection medium (1×MS salts, 3% sucrose, 2 mg/L α-napthaleneacetic acid, 500 mg/L carbenicillin and 100 mg/L kanamycin). Recombinant plants are then regenerated and can be planted in soil for each transformant obtained.

Proteins are harvested from each plant, one transformed with A. tumefaciens containing pBIhGHIn and one transformed with A. tumefaciens containing pBIIchGH using a harvest buffer (50 mM Tris-HCl, pH 8.0, 200 mM NaCl, 5 mM EDTA, and 0.1% Tween 20). These proteins, hGHIn and IchGH can be mixed at 25° C. in a pH 7 buffer and incubated to induce intein splicing in trans between the two fragments. This results in fully formed hGH. The size of the protein can be checked by western analysis using an anti-hGH antibody and the activity of the protein can be checked by growth stimulation of rat Nb2 cells as described previously (Staub, et. al., 2000).

### Example 2

### Production of Human Erythropoietin in Tobacco Plants Using Intein Modification

The activity and structure of human Erythropoietin has been studied in detail (Lai, et al., 1986) and several forms are currently licensed for therapeutic intervention in anemia. The native form of human erythropoietin has been previously expressed in plants (Cheon, et. al., 2004). Expression or erythropoietin in its native form affected plant morphology and reproductive capabilities, hence intein modification may reduce the negative effects of expressing the protein in plants.

This example describes the procedure for producing human erythropoietin (hEPO) from an intein-modified hEPO gene in tobacco, although other plant hosts could be used. In this example we have selected EPO gene (GenBank Accession # NM000799) [SEQ ID NO: 5] and a version of the RecA intein from Mycobacterium tuberculosis (GenBank Accession # X58485) [SEQ ID NO: 6] with the homing endonuclease coding region removed (base pairs 313-1002 of the coding region, corresponding to amino acids 105-334). Variations in each of the different steps can be used to practice this invention, however, the procedure of this example proceeds by: 1) creating the intein modified hEPO gene, 2) packaging the gene into appropriate expression vectors, 3) transforming tobacco with the expression vector, and 4) selecting and regenerating fully developed tobacco plants that express the intein-modified gene. From these plants the intein-modified proteins can be recovered and induced to splice, thereby reconstituting actively formed hEPO.

Design of an intein-modified hEPO gene requires splitting the HEPO coding sequence immediately before the native cysteine at position 29 (Cys29) of the mature peptide. Although other cysteine residues can be used, they result in less efficient splicing after purification. The HEPO gene can be synthesized (Blue Heron, Bothell, Wash.) using the sequences described above, or prepared as described previously (Gangopadhyay, et. al. 2003).

Once synthesized, the intein modified hEPO gene (now referred to as IC29hEPO) [SEQ ID NO: 7] is cloned into the Agrobacterium tumefaciens binary expression vector pPEV-1 (Clontech, CA, USA) as described previously (Cheon, et. al. 2004) to create pPEV-IC29hEOP. PPEV-IC29hEPO was then introduced into A. tumefaciens strain EHA105 by electroporation. Transformed colonies were then selected on YEP medium supplemented with 25 mg/L rifampicin and 50 mg/L kanamycin.

For transformation, leaf sections from nine week old tobacco leaf sections can be used. Following sodium hypochloride sterilization of the leaf sections, each 0.5 cm² section is incubated with the recombinant A. tumefaciens EHA105 containing PEV-IC29hEPO for three days in the dark at 28° C. The incubation occurs for 2-3 days at 28° C. in transformation medium (1× MS salts). Leaf sections are washed with 1×MS containing 250 mg/L carbenicillin and transferred to selection medium (1× MS salts, 3% sucrose, 2 mg/L benzylaminopurine, 0.1 mg/L napthaleneacetic acid, 250 mg/L carbenicillin and 100 mg/L kanamycin). Recombinant shoots formed in the presence of kanamycin are removed and placed on fresh regeneration medium. Recombinant plants are then regenerated by transferring the shoots to rooting medium (1× MS agar, 250 mg/L carbenicillin, 100 mg/L kanamycin) and planting subsequent transformants.

Proteins can be harvested from the plants using a harvest buffer (50 mM Tris-HCl, pH 8.0, 200 mM NaCl, 5 mM EDTA, and 0.1% Tween 20) or other method know in the art. These proteins, IC29hEPO, can be induced to splice by resuspending them in a solution containing 1 mM EDTA, 0.5 M L-arginine, 1 mM DTT (or TCEP), 20 mM sodium phosphate pH 7.5, and 0.5 NaCl and incubating them overnight at 25° C. as described previously (Gangopadhyay et. al., 2003). This results in fully formed hEPO with the intein spliced out. The size of the protein can be checked by western analysis using an anti-hEPO antibody.

The invention now being fully described, it will be apparent to one of ordinary skill in the art that many changes and modifications can be made thereto without departing from the spirit or scope of the invention as set forth herein.

### BIBLIOGRAPHY

Dale, Biotechnol. Prog. 15:775-776 (1999).
Park, et al., Biotechnol. Prog. 14:699-704 (1998).
Taylor, et al., Biotechnol. Prog. 16:541-547 (2000).
Poirier, Nature Biotechnology 17:960-961 (1999).
Ingram, Biotechnol. Prog. 15:855-866 (1999).
Mansfield, et al., Biotechnol. Prog. 15:804-816 (1999).
Evans, et al., Protein Science 7:2256-2264 (1998).
Perler, et al., Nucl. Acids Res. 22:1125-1127 (1994).
Xu, et al., EMBO 15:5146-5153 (1996).
Wood, et al., Biotechnol. Prog. 16:1055-1063 (2000).
Clarke, P.N.A.S. (USA) 91:11084-11088 (1994).
Derbyshire, et al., P.N.A.S. (USA) 95:1356-1357 (1998).
Perler, et al., Nucleic Acids Res. 22:1125-1127 (1994).
Wallace, C. J., Protein Sci. 2:697-705, (1993).
Xu, et al., Cell 75:1371-1377 (1993).
Pietrokovski, S., Protein Sci. 3735:2340-2350 (1994).
Ausbel, et al., “Current Protocols in Molecular Biology,” Wiley, New York (1998).
Draper, et al., “Plant Genetic Transformation and Gene Expression: A Laboratory Manual,” Blackwell Scientific Publications, Boston (1998).
Potrykus, et al., “Gene Transfer to Plants,” Springer, N.Y. (1995).
Guilley, et al., Cell, 30:763 (1982).
Broothaerts, et al., Nature, 433:629 (2005).
Higgins, T. J. V., Annu. Rev. Plant Physiol. 35:191 (1984).
Coruzzi, et al., EMBO J. 3:1671 (1984).
Tingey, et al., EMBO J. 6:3565 (1987).
Ryan, et al., Nuc. Acids Res. 17:3584 (1989).
Rocha-Sosa, et al., EMBO J. 8:23 (1989).
Wenzler, et al., Plant Mol. Biol. 12:41 (1989).
Bird et al., Plant Mol. Biol. 11:651 (1988).
Brederode, et al., Nucl. Acids Res. 8:2213 (1980).
Smeekens, et al., T.I.B.S. 15:73 (1990).
van den Broeck et al., Nature 313:358 (1985).
Schreier, et al., EMBO J. 4:25 (1985).
Tague, et al., Plant Phys. 86:506 (1988).
Von Heijne, G., J. Mol. Biol. 189:239 (1986).
Sijmons, et al., Bio/Technol. 8:217 (1990).
Gordon-Kamm, et al., The Plant Cell 2:603 (1990).
Shimamoto, et al., Nature 338:274 (1989).
Horsch, et al., Science 2:1229 (1985).
Ziegler, et al., Molecular Breeding 6:37-46 (2000).
Dai, et al., (a), Molecular Breeding 6:277-285 (2000).
Dai, et al., (b), Molecular Breeding 9:43-54 (2000).
Montvalvo-Rodriguez, et al., Biotech. and Bioeng. 2:151-159 (2000).
U.S. Pat. No. 6,022,846.
Chu, et al., Sci. Sin. 18:659-668 (1975).
Klein, et al., Nature 327:70-73 (1987).
Chin, et al., PNAS 100:4510 (2003).
Staub, et al., Nature Biotech 18:333 (2000).
Lai et. al., J. Biol. Chem., 261:3116 (1986).
Cheon et. al., Transgenic Research, 13:541 (2004).
Gangopadhyay et. al., Biochimica et Biophysica Acta, 1619:193 (2003).

1. A recombinant plant or plant part, plantlet, tissue, cell, sub-cellular fraction, seed, seedling, protoplast, progeny or descendant, comprising at least one expression construct that encodes at least one modified protein, the at least one expression construct comprising at least one exogenous gene sequence encoding at least one therapeutic protein, the at least one exogenous gene sequence being fused to at least one sequence encoding at least one intervening protein sequence or segment thereof.

2. The recombinant plant or plant part, plantlet, tissue, cell, sub-cellular fraction, seed, seedling, protoplast, progeny or descendant of claim 1, wherein the at least one exogenous gene sequence is divided into multiple segments, each segment being fused to the at least one sequence encoding the at least one intervening protein sequence or segment thereof.

3. A set of recombinant plants or plant parts, plantlets, tissues, cells, sub-cellular fractions, seeds, seedlings, protoplasts, progeny or descendants as claimed in claim 2, wherein each plant or plant part, plantlet, tissue, cell, sub-cellular fraction, seed, seedling, protoplast, progeny or descendant expresses a different segment of the at least one exogenous gene sequence.

4. The recombinant plant, or plant part, plantlet, tissue, cell, sub-cellular fraction, seed, seedling, protoplast, progeny or descendant of claim 1, wherein the at least one therapeutic protein is fused to at least one of the amino terminal end and the carboxyl terminal end of the at least one intervening protein sequence or segment thereof.

5. The recombinant plant, or plant part, plantlet, tissue, cell, sub-cellular fraction, seed, seedling, protoplast, progeny or descendant of claim 1, wherein the at least one exogenous gene sequence is a partial exogenous gene sequence.

6. The recombinant plant, or plant part, plantlet, tissue, cell, sub-cellular fraction, seed, seedling, protoplast, progeny or descendant of claim 1, wherein the at least one expression construct is suitable for protein expression.

7. The recombinant plant, or plant part, plantlet, tissue, cell, sub-cellular fraction, seed, seedling, protoplast, progeny or descendant of claim 1, wherein the at least one expression construct is fused to at least one of a regulatory sequence and selection sequence.

8. The recombinant plant, or plant part, plantlet, tissue, cell, sub-cellular fraction, seed, seedling, protoplast, progeny or descendant of claim 7, wherein the at least one regulatory sequence further comprises at least one of a promoter sequence, an origin of replication sequence, and a signaling sequence.

9. The recombinant plant, or plant part, plantlet, tissue, cell, sub-cellular fraction, seed, seedling, protoplast, progeny or descendant of claim 7, wherein the at least one selective sequence further comprises at least one of a herbicidal gene, an antibacterial gene, a fluorescent maker, a dye marker, and a selectable marker.

10. The recombinant plant, or plant part, plantlet, tissue, cell, sub-cellular fraction, seed, seedling, protoplast, progeny or descendant of claim 9, wherein the at least one selectable marker confers resistance to a chemical comprising at least one of bromoxynil, 2,2-dichloropropionic acid, G418, glyphosphate, haloxyfop, hygromycin, imidazoline, kanamycin, methotrexate, neomycin, phosphinothricin, sethoxydim, 2,2-dichloropropionic acid, trichothecne, sulfonylurea, s-triazine, and triazolopyrimidine.

11. The recombinant plant, or plant part, plantlet, tissue, cell, sub-cellular fraction, seed, seedling, protoplast, progeny or descendant of claim 10, which is resistant to normally toxic levels of at least one chemical.

12. The recombinant plant, or plant part, plantlet, tissue, cell, sub-cellular fraction, seed, seedling, protoplast, progeny or descendant of claim 1, wherein the at least one expression construct is expressed constitutively.

13. The recombinant plant, or plant part, plantlet, tissue, cell, sub-cellular fraction, seed, seedling, protoplast, progeny or descendant of claim 1, wherein the at least one expression construct is expressed transiently.

14. The recombinant plant, or plant part, plantlet, tissue, cell, sub-cellular fraction, seed, seedling, protoplast, progeny or descendant of claim 1, wherein the at least one expression construct is expressed throughout the entire plant.

15. The recombinant plant, or plant part, plantlet, tissue, cell, sub-cellular fraction, seed, seedling, protoplast, progeny or descendant of claim 1, wherein the at least one expression construct is expressed in specific plant tissues.

16. The recombinant plant, or plant part, plantlet, tissue, cell, sub-cellular fraction, seed, seedling, protoplast, progeny or descendant of claim 1, wherein the at least one expression construct is expressed at a pre-determined point of the plant life-cycle.

17. The recombinant plant, or plant part, plantlet, tissue, cell, sub-cellular fraction, seed, seedling, protoplast, progeny or descendant of claim 1, wherein the at least one expression construct is expressed in at least one sub-cellular compartment.

18. The recombinant plant, or plant part, plantlet, tissue, cell, sub-cellular fraction, seed, seedling, protoplast, progeny or descendant of claim 1, wherein the at least one expression construct is expressed and secreted extracellularly.

19. The recombinant plant, or plant part, plantlet, tissue, cell, sub-cellular fraction, seed, seedling, protoplast, progeny or descendant of claim 1, wherein the at least one expression construct is expressed and at least one stimuli triggers splicing of the at least one modified protein.

20. The recombinant plant, or plant part, plantlet, tissue, cell, sub-cellular fraction, seed, seedling, protoplast, progeny or descendant of claim 19, wherein the at least one stimuli comprises at least one of a change in pH, change in osmolality, change in temperature, addition of a pesticide, addition of a chemical, change in light, and change in sound.

21. The recombinant plant, or plant part, plantlet, tissue, cell, sub-cellular fraction, seed, seedling, protoplast, progeny or descendant of claim 1, wherein a stimuli triggers splicing of the at least one modified protein at least one end of the at least one intervening protein sequence or segment thereof.

22. The recombinant plant, or plant part, plantlet, tissue, cell, sub-cellular fraction, seed, seedling, protoplast, progeny or descendant of claim 1, wherein the at least one modified protein is expressed through at least one of a chemical and biological induction.

23. The recombinant plant, or plant part, plantlet, tissue, cell, sub-cellular fraction, seed, seedling, protoplast, progeny or descendant of claim 1, wherein the at least one therapeutic protein is one of a hormone, growth factor, receptor, ligand, antibody, antigen, enzyme, and vaccine.

24. The recombinant plant, or plant part, plantlet, tissue, cell, sub-cellular fraction, seed, seedling, protoplast, progeny or descendant of claim 1, which is fertile and the at least one modified protein is a heritable protein.

25. An inbred recombinant plant, or plant part, plantlet, tissue, cell, sub-cellular fraction, seed, seedling, protoplast, progeny or descendant of claim 1.

26. A hybrid recombinant plant, or plant part, plantlet, tissue, cell, sub-cellular fraction, seed, seedling, protoplast, progeny or descendant of claim 1.

27. The recombinant plant, or plant part, plantlet, tissue, cell, sub-cellular fraction, seed, seedling, protoplast, progeny or descendant of claim 1, wherein the at least one expression construct encodes at least one splice junction.

28. The recombinant plant, or plant part, plantlet, tissue, cell, sub-cellular fraction, seed, seedling, protoplast, progeny or descendant of claim 1, wherein the at least one therapeutic protein is expressed by one of an animal, virus, and microorganism.

29. The recombinant plant, or plant part, plantlet, tissue, cell, sub-cellular fraction, seed, seedling, protoplast, progeny or descendant of claim 1, wherein the at least one therapeutic protein is an animal therapeutic protein.

30. The recombinant plant, or plant part, plantlet, tissue, cell, sub-cellular fraction, seed, seedling, protoplast, progeny or descendant of claim 29, wherein the at least one therapeutic protein is a human therapeutic protein.

31. The recombinant plant, or plant part, plantlet, tissue, cell, sub-cellular fraction, seed, seedling, protoplast, progeny or descendant of claim 1, wherein the at least one intervening protein sequence is an intein.

32. The recombinant plant, or plant part, plantlet, tissue, cell, sub-cellular fraction, seed, seedling, protoplast, progeny or descendant of claim 1, wherein the at least one intervening protein sequence is a controllable intervening protein sequence.

33. A method of producing a recombinant plant, or plant part, plantlet, tissue, cell, sub-cellular fraction, seed, seedling, protoplast, progeny or descendant comprising:
providing at least one expression construct that encodes at least one modified protein, the at least one expression construct comprising at least one exogenous gene sequence encoding at least one therapeutic protein, the at least one exogenous gene sequence being fused to at least one sequence encoding at least one intervening protein sequence or segment thereof; and
transforming at least one plant, or plant part, plantlet, tissue, cell, sub-cellular fraction, seed, seedling, protoplast, progeny or descendant with the expression construct.

34. The method of claim 33, wherein the at least one exogenous gene sequence is divided into multiple segments, each segment being fused to the at least one sequence encoding the at least one intervening protein sequence or segment thereof.

35. A method for producing a set of recombinant plants or plant parts, plantlets, tissues, cells, sub-cellular fractions, seeds, seedlings, protoplasts, progeny or descendants produced according to the method of 34, wherein each plant or plant part, plantlet, tissue, cell, sub-cellular fraction, seed, seedling, protoplast, progeny or descendant expresses a different segment of the at least one exogenous gene sequence.

36. The method of claim 33, wherein the at least one exogenous gene sequence is a partial exogenous gene sequence.

37. The method of claim 33, wherein the at least one exogenous gene sequence is internally fused to the at least one sequence encoding the at least one intervening protein sequence or segment thereof.

38. The method of claim 33, wherein the at least one exogenous gene sequence is terminally fused to the at least one sequence encoding the at least one intervening protein sequence or segment thereof.

39. The method of claim 33, further comprising regenerating at least one recombinant plant, or plant part, plantlet, tissue, cell, sub-cellular fraction, seed, seedling, protoplast, progeny or descendent, from the at least one recombinant plant, or plant part, plantlet, tissue, cell, sub-cellular fraction, seed, seedling, protoplast, progeny or descendant that encodes the at least one therapeutic protein sequence.

40. The method of claim 39, wherein the regeneration step is conducted by at least one of:
breeding of a recombinant plant, or plant part, plantlet, tissue, cell, sub-cellular fraction, seed, seedling protoplast, progeny or descendent;
crossing of a recombinant plant, or plant part, plantlet, tissue, cell, sub-cellular fraction, seed, seedling, protoplast, progeny or descendent and a non-genetically recombinant plant, or plant part, plantlet, tissue, cell, sub-cellular fraction, seed, seedling, protoplast, progeny or descendent; and
back-crossing of two genetically recombinant plants, or plant parts, plantlets, tissues, cells, sub-cellular fractions, seeds, seedlings, protoplasts, progeny or descendents.

41. The method of claim 33, wherein the at least one expression construct comprises at least one of a promoter, selectable marker, resistance marker, heritable marker, poly-adenylation sequence, repressor, enhancer, localization sequence, and signaling sequence.

42. The method of claim 33, wherein the at least one plant, or plant part, plantlet, tissue, cell, sub-cellular fraction, seed, seedling, protoplast, progeny or descendent is transformed with the at least one expression construct by at least one of viral transformation, bombardment with DNA-coated microprojectiles, liposomal gene transformation, bacterial gene transfer, electroporation, and chemical gene transformation.

43. The method of claim 33, wherein the transformed plant, or plant part, plantlet, tissue, cell, sub-cellular fraction, seed, seedling, protoplast, progeny or descendent is selected with the aid of at least one of a selectable marker and resistance marker.

44. The method of claim 33, wherein the recombinant plant, or plant part, plantlet, tissue, cell, sub-cellular fraction, seed, seedling, protoplast, progeny or descendent is regenerated from at least one of a transformed embryogenic tissue, a plant protoplast, a cell derived from immature embryo, and a transformed seed.

45. The method of claim 33, further comprising cloning the at least one exogenous gene sequence and the at least one sequence encoding the at least one intervening protein sequence or segment thereof.

46. The method of claim 33, wherein the at least one therapeutic protein is an animal therapeutic protein.

47. The method of claim 46, wherein the at least one therapeutic protein is a human therapeutic protein.

48. The method of claim 33, wherein the wherein the at least one intervening protein sequence is an intein.

49. The method of claim 33, wherein the at least one intervening protein sequence is a controllable intervening protein sequence.

50. A method for producing a modified protein, protein segment, or therapeutic protein from at least one recombinant plant, or plant part, plantlet, tissue, cell, sub-cellular fraction, seed, seedling, protoplast, progeny or descendant comprising:
providing at least one expression construct that encodes at least one modified protein, the at least one expression construct comprising at least one exogenous gene sequence encoding at least one parent therapeutic protein, the at least one exogenous gene sequence being fused to at least one sequence encoding at least one intervening protein sequence or segment thereof;
transforming at least one plant, or plant part, plantlet, tissue, cell, sub-cellular fraction, seed, seedling, protoplast, progeny or descendant with the expression construct; and
expressing the at least one modified protein.

51. The method of claim 50, further comprising reforming the at least one therapeutic protein in vitro by:
mixing a set of recombinant plants having the at least one modified protein, the set of recombinant plants expressing different subsequences of the parent therapeutic protein; and
splicing the at least one modified protein to remove the at least one intervening protein sequence portions thereof; and
relegating the resulting therapeutic protein subsequences such that the fully active parent therapeutic protein is recovered.

52. The method of claim 50, further comprising harvesting the at least one modified protein from the recombinant plant, or plant part, plantlet, tissue, cell, sub-cellular fraction, seed, seedling, protoplast, progeny or descendant.

53. The method of claim 50, further comprising purifying the at least one modified protein.

54. The method of claim 50, further comprising regenerating at least one recombinant plant, or plant part, plantlet, tissue, cell, sub-cellular fraction, seed, seedling, protoplast, progeny or descendent, from the at least one recombinant plant, or plant part, plantlet, tissue, cell, sub-cellular fraction, seed, seedling, protoplast, progeny or descendant that encodes the at least one parent therapeutic protein sequence.

55. The method of claim 50, wherein the at least one intervening protein sequence or segment thereof is capable of effecting at least one of excision, cleavage, ligation, excision-ligation, cleavage-ligation, and cyclization.

56. The method of claim 50, wherein two or more therapeutic proteins are produced in one of a transgenic plant and set of transgenic plants.

57. The method of claim 50, wherein the at least one therapeutic protein is an animal therapeutic protein.

58. The method of claim 57, wherein the at least one therapeutic protein is a human therapeutic protein.

59. The method of claim 50, wherein the wherein the at least one intervening protein sequence is an intein.

60. The method of claim 50, wherein the at least one intervening protein sequence is a controllable intervening protein sequence.

61. A process for making therapeutic proteins from plants expressing at least one modified protein comprising:
dividing a nucleic acid coding sequence of at least one therapeutic protein into multiple segments;
fusing each segment with at least one sequence encoding an intervening protein sequence or segment thereof;
constructing multiple expression vectors that allow for the expression of the at least one modified protein sequence;
genetically modifying a set of plants with each of the expression vectors previously formed such that the entire coding sequence of the at least one therapeutic protein is contained within the set of plants;
growing the genetically modified plants;
harvesting the genetically modified plants;
expressing the at least one modified protein; and
milling the genetically modified plants and combining them in a manufacturing process wherein the at least one intervening protein sequence or segment thereof is induced to splice from the at least one modified protein and reforming and recovering the at least one resulting therapeutic protein.

62. The process of claim 61, wherein the expression vector further allows for the selection of plants transformed with the vector.

63. The process of claim 61, further comprising purification of the at least one therapeutic protein.

64. The process of claim 61, wherein the manufacturing process is one of a chemical and mechanical manufacturing process.

65. The method of claim 61, wherein the at least one therapeutic protein is an animal therapeutic protein.

66. The method of claim 65, wherein the at least one therapeutic protein is a human therapeutic protein.

67. The method of claim 61, wherein the wherein the at least one intervening protein sequence is an intein.

68. The method of claim 61, wherein the at least one intervening protein sequence is a controllable intervening protein sequence.


SEQUENCE LISTING (7 sequences)

SEQ ID NO: 1 - DNA, 582 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 2 - DNA, 609 bp, Synechocystis sp.
atggctttcccgactattccactgagccgcctgttcgacaacgctatgctgcgagctcaccgtctgcaccagctggctttcgacacctaccaggagttcgaagaagcatacatcccgaaagaacagaaatactccttcctgcagaacccgcagacctccctgtgcttctccgaaagcattccgaccccgagcaaccgcgaagaaacccagcagaaatccaacctcgagctgctgcgtatctgcctttctttcggaactgagatccttaccgttgagtacggaccacttcctattggtaagatcgtttctgaggaaattaactgctcagtgtactctgttgatccagaaggaagagtttacactcaggctatcgcacaatggcacgataggggtgaacaagaggttctcgagtacgagcttgaagatggatccgttattcgtgctacctctgaccatagattcttgactacagattatcagcttctcgctatcgaggaaatctttgctaggcaacttgatctccttactttggagaacatcaagcagacagaagaggctcttgacaaccacagacttccattccctttgctcgatgctggaaccatcaag

SEQ ID NO: 3 - DNA, 453 bp, Synechocystis sp.
atggttaaggtgattggaagacgttctcttggtgttcaaaggatcttcgatatcggattgccacaagaccacaactttcttctcgctaatggtgccatcgctgccaattgctccctgctgctgattcaatcctggctggaaccagttcagttcctgcgttccgttttcgctaactcactagtttatggcgcttccgactccaacgtttacgacctgctgaaggacctggaagaaggtattcagactctgatgggtcgtctggaggacggttcccctaggaccggtcagatcttcaaacagacctatagcaaattcgacaccaactcccacaacgacgacgcgttgctgaaaaactacggtctgctgtactgcttccggaaggacatggacaaggttgagactttcctgcgtatcgttcagtgccgttccgttgaaggttcctgcggtttctaa

SEQ ID NO: 4 - DNA, 8196 bp, Artificial
  Agrobacterium tumefaciens binary expression vector pBI121
aaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagcccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagagtttgaaattctctgtgaattttttttgttgattgatgattgtcttcaaaaacattcctgggtttgacttttttttcatcggagcttcgtctgtgttgttggttgatgtcaattggtatttgtattaatccgtactttttcaatgattgctttcttaagttttttaacatttgttttttggcggcggagaaatctggagtaatgttgaaaaacaatgacaattttgtttctttcaatcctaaattttcgatttttctcactacccttatctctatttgatcgattttttgttattgtttacagatgaaaaagcagagaattgttgtgaaaatcgggagcagctcgctcacaaacagtaacggaagtattgatgaagccaaaatcaaggagcatacggaagccatttcattattaaaagaaaacgggcatgaagtaatcctcattacctcaggcgcggtggcagccggtttttccgctctcggctatccggcgcgccccgttacgataaaaggaaagcaggcagccgccgcggtcggccagacgcttttaatgcagcaatatatggaccatttaaaaagatacggtctgacgccggcgcaaattttattaacaagaaatgatttctcaaaaagagagcggtacagaaatgcgtatgccacggtaatggaattattggagcggggagtcgtgccgattattaatgagaatgattccacatcagtcgaagaactgacgttcggagataatgacatgctgtcggcgcttgtgagcggcctgattcatgcagaccaattaatgattctcacagacattaacgggctgtacgatgccaatccgaatgaaaaccctgacgcaaagcggtttgactatttgactgacattacacctgaattgctcgggtatgccggatcagcgggctcgaaggtcggcaccggaggcatgaagtcaaaatttctcgctgcccagaccgcattgtcgctcggagttaaggtgtttatcggaaccggtgccggacgggaaaaactgaaggtgattttagacggtaaaggcgacggcacatatatcggtgataaagagctgtccaccgtcaataatacacggcaatggatcatgtttcattctccgatatcaggggaaatcatcattgatgggggagcggagcaggcgatgatccataacggctcaagtctccttccggccggtgtcgccgatgtctgcggcagttttccgaaaggggccgtggtggaagtcagaggtcccggcggcatcatcggcaaagggcagacgcattactcatccgatgagattttggaagccaaagggaagcggagcgatcagcttccgggcgcgaagcaggtggaagtgattcacagaaatgattgggtgaatctatttgacgagggggaaaacaaatgatctagaggatccgtttgaaattctctgtgaattttttttgttgattgatgattgtcttcaaaaacattcctgggtttgacttttttttcatcggagcttcgtctgtgttgttggttgatgtcaattggtatttgtattaatccgtactttttcaatgattgctttcttaagttttttaacatttgttttttggcggcggagaaatctggagtaatgttgaaaaacaatgacaattttgtttctttcaatcctaaattttcgatttttctcactacccttatctctatttgatcgattttttgttattgtttacagatgagtgaagtattgcaaaaagctgccttggcaaaagaagcggctgccgaaatggtcatgaaaacgactgctgaaaaaaacgaagcgctccaatgtattgcagacggactcagaaatgaacgccagctgattttaacagagaatcagaaagatattgaagcgggacagaaaagagggctgacacctgacattattgacaggctgactcttgatgaaaaacgcctgctcgatatcgcggatgctgtagaactgttaatcggtctggaagatcccgtcggtgaatcattggagaccattcaaaaagagaatggattatccattgaaaaaatccgcgtgcccctcggagtagtgggcatgatctatgaagccagaccgaatgttaccgtagacgctgccacactttgcctgaaaaccggaaacgcggttgtgctcagaggaagttcgtcagccatccacagcaacaaagcgcttgtcagtgtgatgaaacgcgcactgggactgtcaaagcttccgattgatgccgttcagctcatagaagatacaagcaaagaaaccgcgaaacagctcttcacattaaatgacgggctggatgtattgatcccgcgcggcggcaaaaatttgattgatctggtggtcagggaatcaaccgtacccgtgttagaaaccggagcgggcaactgccacgtgtacatagatgagtcagcagatcccgatatggcaagagatgtcgtcatcaacgccaaaacacagcggccttctgtctgcaatgcgattgaatctcttttgattcatgaaaaatgggcggaagtacacggacgaaagctgctgaatcagctgacagaaaaaggggtggagctacgcggagatcagatgatttgcgagctggagcctaatgcgaaacaagccgaagaggccgattggggagcggaatacctggcgccgattctcagtgtaaaaacggtgcatgacgtacaggaggcggtccgccacattcagcagtacggcacaaaccattcagaagccattctgacggaaaacggtgagcatgccgcttacttccagacagccgttgacgccgccgccgtgtatcataatgcttccacacggtttacagacggctttgaattctgaggatccccgggtggtcagtcccttatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcag

SEQ ID NO: 5 - DNA, 582 bp, Homo sapiens; identical to SEQ ID NO: 1

SEQ ID NO: 6 - DNA, 636 bp, Mycobacterium tuberculosis
tgcctcgcagagggcactcggatcttcgatccggtcaccggtacaacgcatcgcatcgaggatgttgtcgatgggcgcaagcctattcatgtcgtggctgctgccaaggacggaacgctgcatgcgcggcccgtggtgtcctggttcgaccagggaacgcgggatgtgatcgggttgcggatcgccggtggcgccatcgtgtgggcgacacccgatcacaaggtgctgacagagtacggctggcgtgccgccggggaactccgcaagggagacagggtggcgcaaccgcgacgcttcgatggattcggtgacctggctgcagagatgaccgatgccgtgctgaattatctggacgagcgcggcgtgaccgcgcaggaggccgcggccatgatcggtgtagcttccggggacccccgcggtggaatgaagcaggtcttaggtgccagccgccttcgtcgggatcgcgtgcaggcgctcgcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgtcgtgcacaac

SEQ ID NO: 7 - DNA, 1137 bp, Artificial
  Intein modified human erythropoietin gene (IC29hEPO)
gccccaccacgcctcatctgtgacagccgagtcctggagaggtacctcttggaggccaaggaggccgagaatatcacgacgggctgcctcgcagagggcactcggatcttcgatccggtcaccggtacaacgcatcgcatcgaggatgttgtcgatgggcgcaagcctattcatgtcgtggctgctgccaaggacggaacgctgcatgcgcggcccgtggtgtcctggttcgaccagggaacgcgggatgtgatcgggttgcggatcgccggtggcgccatcgtgtgggcgacacccgatcacaaggtgctgacagagtacggctggcgtgccgccggggaactccgcaagggagacagggtggcgcaaccgcgacgcttcgatggattcggtgacctggctgcagagatgaccgatgccgtgctgaattatctggacgagcgcggcgtgaccgcgcaggaggccgcggccatgatcggtgtagcttccggggacccccgcggtggaatgaagcaggtcttaggtgccagccgccttcgtcgggatcgcgtgcaggcgctcgcggatgccctggatgacaaattcctgcacgacatgctggcggaagaactccgctattccgtgatccgagaagtgctgccaacgcggcgggcacgaacgttcgacctcgaggtcgaggaactgcacaccctcgtcgccgaaggggttgtcgtgcacaactgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga